(19) United States
(12) Patent Application Publication
(10) Pub. No.: US 2019/0017988 A1
(43) Pub. Date: Jan. 17, 2019

(54) MICROELECTRODE AND MICROELECTRODE ARRAY FOR DETECTING, RECORDING, STIMULATING OR MONITORING ACTIVITY OF ELECTRICALLY EXCITABLE CELLS

(71) Applicant: UTI LIMITED PARTNERSHIP, Calgary (CA)

(72) Inventors: Pierre WIJDENES, Calgary (CA); Colin DALTON, Calgary (CA); Naweed I. SYED, Calgary (CA)

(73) Assignee: UTI LIMITED PARTNERSHIP, Calgary, AB (CA)

(21) Appl. No.: 16/069,096

(22) PCT Filed: Jan. 12, 2017
(86) PCT No.: PCT/CA2017/050041

§ 371 Date: Jul. 10, 2018

Related U.S. Application Data
(60) Provisional application No. 62/277,803, filed on Jan. 12, 2016.

(51) Int. Cl. G01N 33/483; C12M 1/34; H01B 5/14; G06N 3/10

(52) U.S. Cl. CPC G01N 33/4836; C12M 41/46; H01B 13/0036; G06N 3/10; H01B 5/14

(57) ABSTRACT

A microelectrode or an array of microelectrodes for communicating with one or more adjacent electrically excitable cells. The microelectrode array comprises two or more microelectrodes. Each microelectrode comprises a body with a perimeter; an electrode wire that is electronically connected to the body and that is electronically connectible to an electronic system; and a ridge that extends away from the perimeter of the body for increasing a sealing-resistance value between the electrode and the one or more adjacent electrically excitable cells.

### TECHNICAL FIELD

The present disclosure relates to the field of microelectrodes. In particular, the present disclosure relates to microelectrodes that are arranged as individual electrodes or in arrays for detecting, recording, stimulating, and monitoring activity of individual or synaptically connected electrically excitable cells.

### BACKGROUND

Advances in micro-scale and nano-scale fabrication processes have considerably influenced the development of biomedical devices, including neuro-electronic hybrid devices (NEHDs). NEHDs allow for both recording electrical activity from electrically excitable cells as well as stimulating one or more electrically excitable cells. NEHDs have opened additional avenues to explore fundamental biological and electrophysiological principles.

Micro-scale and nano-scale NEHDs are known to offer the ability to study neural connectivity, network activity, sub-threshold potentials, and brain plasticity, amongst other applications. Some forms of these NEHDs include penetrating and non-penetrating nanopillar electrodes, carbon-nanotube electrodes, mushroom-shaped protruding microelectrodes and planar-microelectrode arrays (MEAs). Efforts have been made to modify these devices to improve coupling between the electrodes and cultured cells in-vitro. These NEHDs can detect action potentials, but rarely can they detect sub-threshold currents that require a resolution lower than intracellular recording methods such as sharp electrodes or whole-cell patch-clamp electrodes. This detection by the NEHDs can occur while maintaining stable contact and with a potential for recording over extended periods of time.

One type of known NEHD is a three-dimensional microelectrode array (3D-MEA). 3D-MEAs are able to record neural activity with a higher resolution than other known devices, such as traditional planar MEAs. However, the higher resolution 3D-MEAs are only able to obtain neural recordings for about 2 days. This limited application renders 3D-MEAs inappropriate for longer-term studies, for example studies of cellular network phenomena such as neural network formation and plasticity.

### SUMMARY

Some embodiments of the present disclosure relate to individual planar microelectrodes, which can also be arranged into microelectrode arrays (MEAs). The microelectrodes can establish one or two way communication with an electrically excitable cell. In some embodiments of the present disclosure, this communication includes one or more of detecting, recording, stimulating or otherwise monitoring of the electrical activity of an electrically excitable cell. The microelectrodes of the present disclosure include a ridge that provides a cell-coupling coefficient that is many times higher than a traditional, planar MEA that does not include a ridge.

In contrast to the known three dimensional microelectrode arrays (3D-MEA), embodiments of the present disclosure relate to microelectrodes that may communicate with an electrically excitable cell for time-periods that may be similar to the time-periods reported with traditional, planar MEAs. For example, some embodiments of the present disclosure relate to microelectrodes that can maintain communication with an electrically excitable cell for at least one month and more.

Some embodiments of the present disclosure relate to microelectrodes that can record neural activity with a cell-coupling coefficient that is about 15 times higher than a traditional planar microelectrode, or potentially higher given different cell types and positioning on the microelectrode.

One embodiment of the present disclosure relates to a microelectrode for communicating with an electrically excitable cell. The microelectrode may comprise a body with perimeter; an electrode wire that is electronically connected to the body and that is electronically connectible to an electronic system. The electrode further comprises a ridge that extends away from the perimeter of the body. The ridge may increase a sealing resistance value between the electrode and the electrically excitable cell. More generally, this microelectrode can communicate with one or more electrically excitable cells by detecting, recording, stimulating and monitoring ionic fluxes across the cell membrane of the one or more electrically excitable cells.

Another embodiment of the present disclosure relates to a MEA for communicating with one or more portions of one or more electrically excitable cells. The microelectrode array may comprise two or more microelectrodes. Each microelectrode comprises a body with a perimeter and an electrode wire that is electronically connected to the body and is electronically connectible to an electronic system. Each electrode further comprises a ridge that extends away from the perimeter of the body. The ridge may increase a sealing-resistance value between the electrode and the one or more portions of the one or more electrically excitable cells. More generally, these microelectrodes of the MEA can communicate with one or more electrically excitable cells by detecting, recording, stimulating and monitoring ionic fluxes across the cell membrane of the one or more electrically excitable cells.

The embodiments of the present disclosure may fill a technological gap by linking the advantages of traditional planar microelectrodes and 3D microelectrodes. The presence of a nano-scale ridge enables the recording of the electrical activity at a portion of an electrically excitable cell at a resolution that is similar to or higher than most 3D microelectrodes, while permitting continuous recording over a month or more causing little or no damage to the electrically excitable cell. Without being bound by any particular theory, it was postulated that bridging the gap between traditional, planar microelectrodes and 3D microelectrodes may provide tools to investigate biological-phenomena, including those comprised of the neural system, such as, but not limited to: neural network formation, neural plasticity, neural dysfunction, long-term effects of the local environment on individual cells, long term effect of the local environment on neuron networks, drug screening and/or the diagnosis of disease. These tools may also detect sub-threshold currents. In addition, a computer model simulation revealed the physical phenomena behind the effect of the nano-scaled ridge: an increase in the sealing resistance value is observed if the cell's diameter is at least equal to the electrode's diameter.

Embodiments of the present disclosure may increase the cell-coupling coefficient between a microelectrode and an electrically excitable cell by a factor of at least 15. Higher cell-coupling coefficients may depend on the location upon the electrically excitable cell an interface is established with the microelectrode, adherence of the electrically excitable cell to the microelectrode and the nature of the local environment, such as the extra-cellular milieu.

Embodiments of the present disclosure relate to microelectrodes that dramatically increased the amplitudes of detected electrical signal from electrically excitable cells from less than about 1 milli-volt (mV) to about 10.6 mV peak-to-peak with a coupling coefficient from 0.001 to at least 0.15. These results may arise due to the structure of the microelectrodes offering an increased sealing resistance (Rseal). The microelectrodes of the present disclosure may record electrical activity of electrically excitable cells at a resolution that is higher than known devices that utilize 3D microelectrodes, while maintaining a longer-term recording activity.

In some embodiments of the present disclosure, some microelectrode parameters such as the size and spatial pattern of the electrodes, or the types and thickness of materials, may be adjusted to optimize the signal-to-noise resolution while the microelectrodes are fabricated using one or more fabrication processes.

Some embodiments of the present disclosure may provide devices that can establish one-way or two-way communication with electrically excitable cells. This communication may permit the study of electrically excitable cells at a desired resolution and for extended periods of time. Embodiments of the present disclosure may provide opportunities to study how electrically excitable cells communicate over time to study the long-term effects of drugs on electrically excitable cells and their activity.

### BRIEF DESCRIPTION OF THE DRAWINGS

Features of the present disclosure will become more apparent in the following detailed description in which reference is made to the appended drawings:

FIG. 1 is a schematic representation of an example synaptic interface between a pre-synaptic neuron and a post-synaptic neuron;

FIG. 2 is a schematic representation of the physical and electrical interface between a microelectrode according to one embodiment of the present disclosure with a ridge and an electrically excitable cell;

FIG. 3 shows an example of atomic force microscopy analysis of a microelectrode according to one embodiment of the present disclosure: A) is an isometric view of a three dimensional, computed-rendered image of the microelectrode; and B) shows an example of height data of the microelectrode shown in A);

FIG. 4 is a top-plan view of a microelectrode array according to one embodiment of the present disclosure with neural cells cultured thereupon;

FIG. 5 shows an example of recordings of electrical activity of some of the neural cells shown in FIG. 4: A) shows multiple action potentials from a single neural cell; and B) shows the recording of a single action potential from a single neural cell with a higher temporal resolution than shown in A);

FIG. 6 is a bar graph of an example of maximum peak-to-peak action potential amplitudes recorded with different examples of microelectrode arrays;

FIG. 7 is a scatter plot that compares the coupling coefficient versus time of measurable electrical recordings for different types of microelectrode arrays;

FIG. 8 is a schematic representation of a computer model simulation used to characterize a microelectrode according to one embodiment of the present disclosure when interfaced with a neural cell;

FIG. 9 shows example of a heat-map generated using data extracted from the computer model simulation of FIG. 8;

FIG. 10 is a line graph of sealing-resistance values versus ridge height for different neural cell diameters determined using the computer model simulation of FIG. 8;

FIG. 11 is a line graph of example sealing resistance values versus neural cell diameter for ridge heights determined using the computer model simulation of FIG. 8;

FIG. 12 is a schematic representation of the physical and electrical interface between a microelectrode according to another embodiment of the present disclosure and an electrically excitable cell;

FIG. 13 is a schematic representation of the physical and electrical interface between a microelectrode according to another embodiment of the present disclosure and an electrically excitable cell;

FIG. 14 is a schematic representation of the physical and electrical interface between a microelectrode according to another embodiment of the present disclosure and an electrically excitable cell;

FIG. 15 is a schematic representation of the physical and electrical interface between a microelectrode according to another embodiment of the present disclosure and an electrically excitable cell; and

FIG. 16 is a line graph that shows sealing resistance versus ridge width when cell diameter, microelectrode diameter and ridge height are held constant.

### DETAILED DESCRIPTION

Embodiments of the present disclosure relate to a microelectrode with a ridge and microelectrode array (MEA) that comprises at least one microelectrode with a ridge. Electrically excitable cells can be cultured and positioned at the surface of an MEA at the interface with a microelectrode. The ridge is extendible towards the electrically excitable cell in terms of height and width. When the electrically excitable cell is close to or in contact with the microelectrode an interface is formed and the microelectrode may communicate with the electrically excitable cell of interest. The communication may be one-way or two-way, electrical, chemical or both. The communication may be in the form of ionic flux from the electrically excitable cell that is detected by the microelectrode and modifies the electrical potential at the surface of the microelectrode. At least one microelectrode translates the communications into electrical signals that are sent to an electrical system where they are interpreted and recorded. Optionally, the microelectrode may also communicate with one or more electrically excitable cells. Optionally, the microelectrode may be positioned at an interface at different locations of the electrically excitable cell. For example, if the electrically excitable cell is a neuron, the interface may be established at the soma or the interface may be formed elsewhere such as an axon or dendrite and the microelectrode may detect local changes in ionic flux or levels due to propagation of action potentials along the neuron.

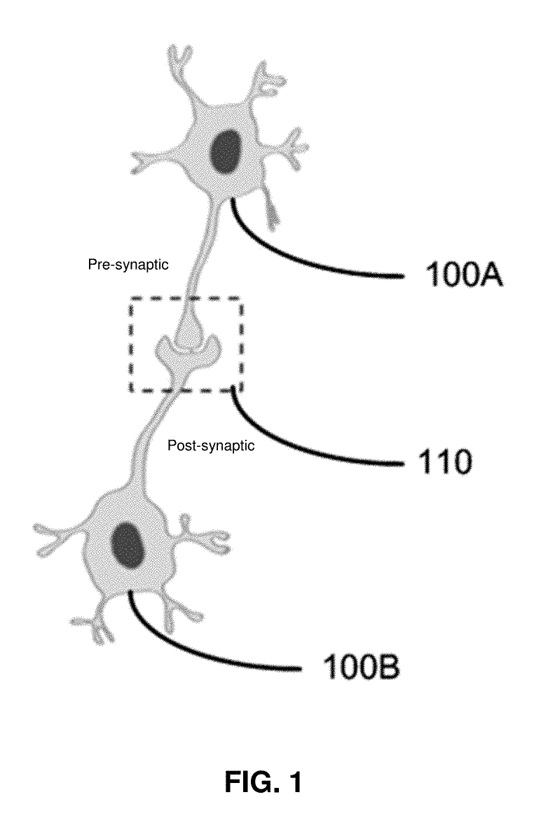

By incorporating the ridge, the microelectrode mimics how a post-synaptic neuron can engulf an axon terminal of a pre-synaptic neuron. FIG. 1 shows an example neuronal synapse 110 (shown within the dashed box of FIG. 1). The synapse is defined by a terminal portion of a pre-synaptic neuron 100A that is at least partially engulfed by a terminal portion of a post-synaptic neuron 100B. Without being bound by any particular theory, it was postulated that this engulfing arrangement of the microelectrode with any portion of a membrane of an electrically excitable cell may contribute towards the strength and quality of communication that is established and exchanged between the microelectrode and the electrically excitable cell.

### Definitions

Unless defined otherwise, all technical and scientific terms used herein have the same meaning as commonly understood by one of ordinary skill in the art to which this disclosure belongs.

As used herein, the term “about” refers to an approximately +/−10% variation from a given value. It is to be understood that such a variation is always included in any given value provided herein, whether or not it is specifically referred to.

As used herein, the term “electrically excitable cells” refers to cells that have the potential to communicate charged ions across the cellular membrane in response to an electric, chemical or physical stimuli. In some instances, the electrically excitable cells can depolarize in a regulated manner to propagate an action potential or end-plate potentials. Some examples of electrically excitable cells include but are not limited to all types of neural cells and muscle cells.

As used herein, the terms “engulf”, “engulfing” and “engulfs” refer to a portion of the microelectrode surrounding, either partially or entirely, at least a portion of an adjacent cell so as to increase the sealing resistance at the interface between an individual microelectrode and the adjacent cell.

As used herein, the term “ridge” describes a topographical feature of the microelectrode described herein. The ridge extends away from a surface of the microelectrode, which can be part of an array or used individually. The ridge may be a continuous or discontinuous feature that is localized towards a perimeter of the electrode. The height and width of the ridge may vary within a given microelectrode and between microelectrodes.

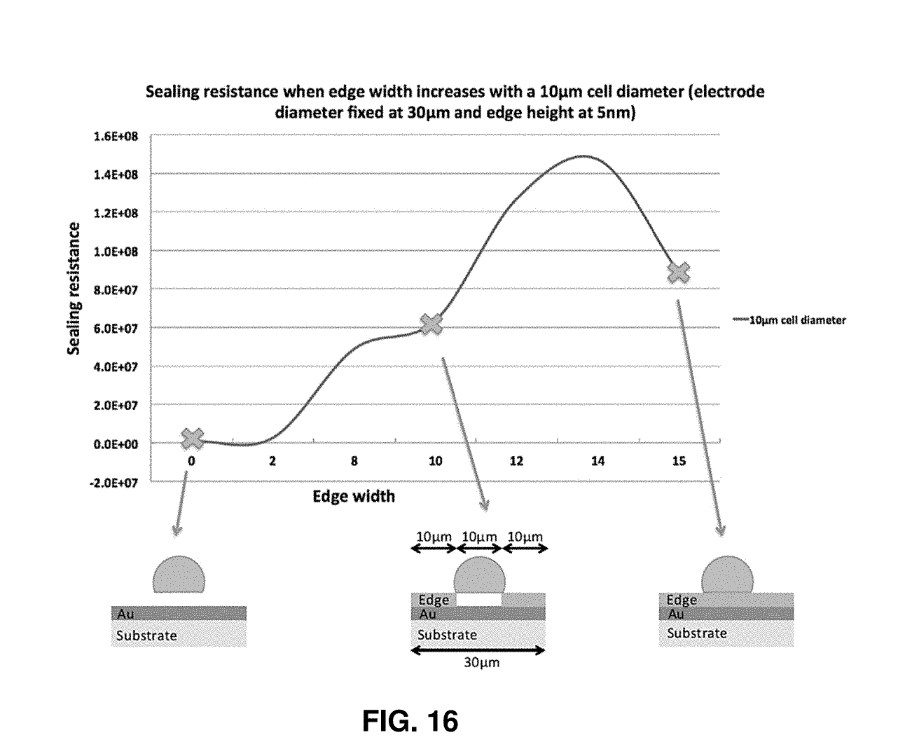

Embodiments of the present disclosure will now be described with reference to FIG. 1 through to FIG. 16, which show representations of embodiments of the present disclosure and examples of data that relate to the function thereof.

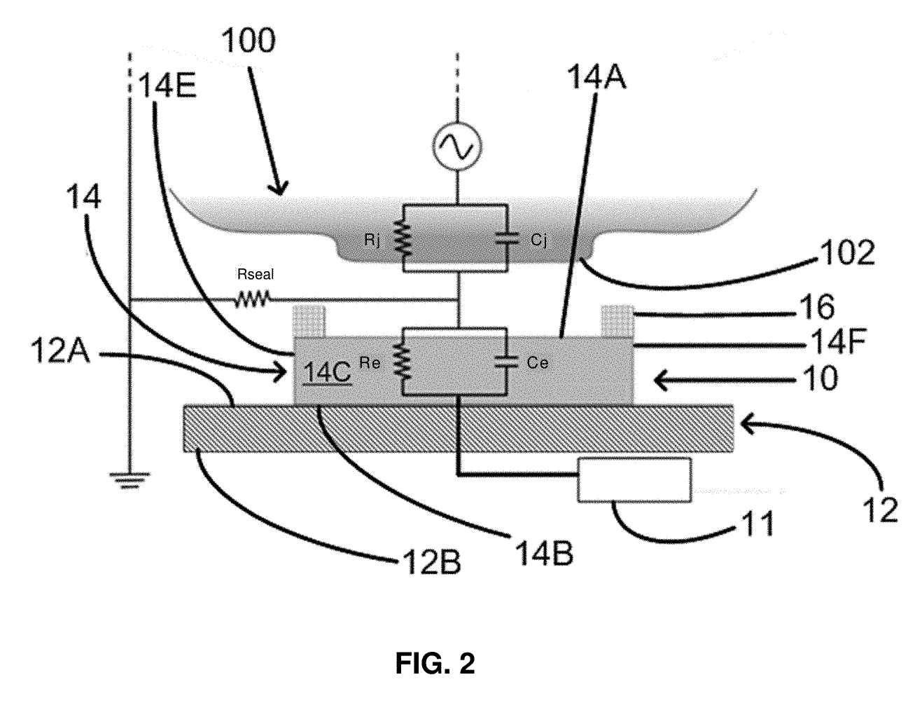

FIG. 2 depicts an interface between one embodiment of a microelectrode 10 and an electrically excitable cell 100, in this case a neural cell. The interface may form a passive, analogue electrical-circuit. The electrically excitable cell 100 may be any type of neural cell including but not limited to: a pre-synaptic neuron, a post-synaptic neuron, a sensory neuron, an interneuron, a motor neuron or a pyramidal neuron. The interface with the neural cell may occur at one or more parts of the neural cell such as, but not limited to: one or more dendrites, the soma, the axon hillock, an axon or one or more axon terminals. FIG. 2 depicts one interface of the microelectrode 10 that is positioned opposite to a junctional membrane 102 within a neural cell body. Via the interface, the microelectrode 10 and the electrically excitable cell 100 can communicate across the gap therebetween. As will be appreciated by one of skill in the art, the interface between the microelectrode 10 and the electrically excitable cell 100 depicted in FIG. 2 may be established in vitro or in vivo.

In the embodiment depicted in FIG. 2, the microelectrode 10 comprises a substrate 12, an electrode base 14 and a ridge 16. The substrate 12 has at least a first surface 12A and a second surface 12B. In one embodiment, the substrate 12 may be a surface of silicon dioxide (glass). The electrode base 14 may be positioned or fabricated upon the first surface 12A of the substrate 12. The electrode base 14 may be planar with a first base surface 14A and a second base surface 14B. The first base surface 14A is in fluid communication with the interface between the microelectrode 10 and an adjacent electrically excitable cell 100. The first base surface 14A may also be referred to as an interfacial surface and the second base surface 14B may be referred to as the substrate surface. A distance between these two base surfaces 14A, 14B defines the height of the electrode base 14. The electrode base 14 may also have opposing sides 14E, 14F. A distance between these sides 14E, 14F may define a width of the electrode base 14 and if the electrode base 14 is substantially circular in shape, from a top perspective view, then the distance between the sides 14E, 14F may define a diameter of the electrode base 14.

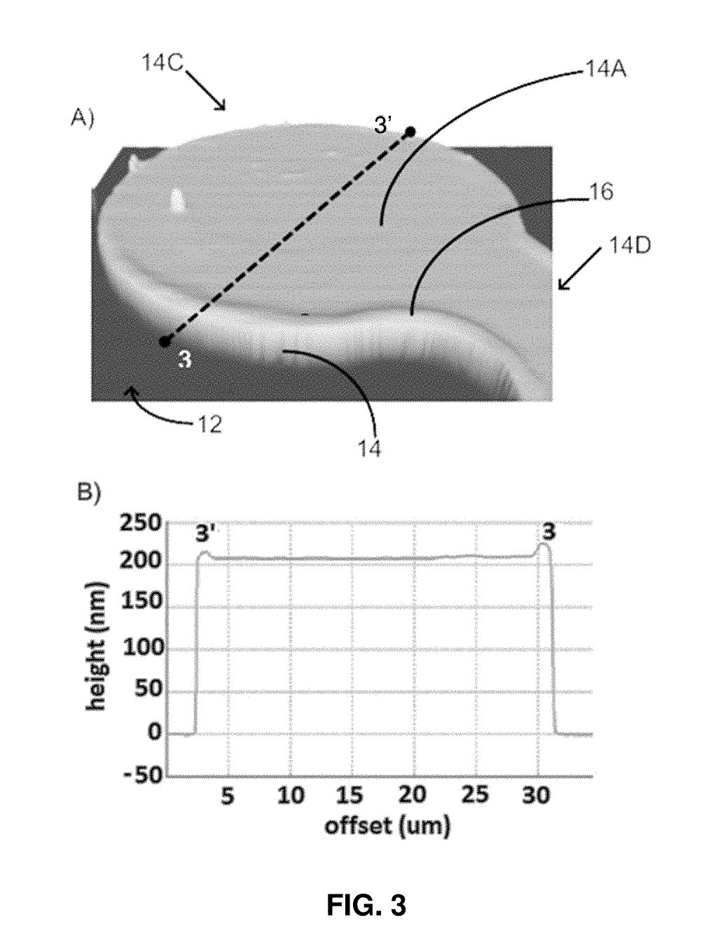

The electrode base 14 may also comprise an electrode body 14C and an electrode wire 14D as depicted in FIG. 3A. The electrode body 14C is configured to communicate across the synaptic cleft with the electrically excitable cell 100. The electrode wire 14D may electronically connect with an electronic system 11 that can be connected to multiple individual electrodes, forming an array.

In an embodiment, the electrode base 14 may have a height of about 1 nm and above. When viewed in top plan view, the electrode body 14C may have one of a variety of geometric shapes including but not limited to substantially non-circular shapes like a star, a triangle, a rectangle, a toms, a square, an ellipse and substantially circular shapes like a spiral and a circle. The geometric shape is defined by an outer perimeter of the electrode body 14C. In one embodiment of present disclosure the electrode base 14 has an electrode body 14C with a substantially circular geometry with a diameter of about 30±1 μm and a height of about 250±15 nm. The diameter and height of the electrode base 14 can vary depending on the intended application which itself depends upon one or more of the shape, size and types of electrically excitable cells with which communication is desired.

The ridge 16 may extend away from the first base surface 14A of the electrode base 14 and also away from the substrate 12. FIG. 2 shows one embodiment of the present disclosure where the ridge 16 extends around the perimeter of the electrode body 14C and the ridge 16 extends upwardly away from the first base surface 14A. In an embodiment of the microelectrode 10 where the electrode body 14C is substantially circular, the ridge 16 may extend above and generally around a circumference of the electrode body 14C. As will be appreciated by those skilled in the art, the ridge 16 may extend above and generally around the perimeter of the electrode body 14C, regardless of the specific geometric-shape of the electrode body 14C. The ridge 16 may extend above either or both of the electrode body 14C and the electrode wire 14D. One skilled in the art will appreciate that the terms “upwardly” and “above” are used herein only in reference to the configuration shown in FIG. 2. The ridge 16 may extend in any direction away from the first base surface 14A so that the ridge 16 engulfs at least one portion of the electrically excitable cell 100, as discussed further below.

The ridge 16 may extend above the first base surface 14A with a substantially consistent height or a variable height. In one embodiment of the present disclosure, the ridge 16 may have a substantially consistent height of between about 5 nm and about 15 nm and a width of about 1 μm to about 4 μm. The height and width of the ridge 16 can vary from these ranges depending upon the intended application.

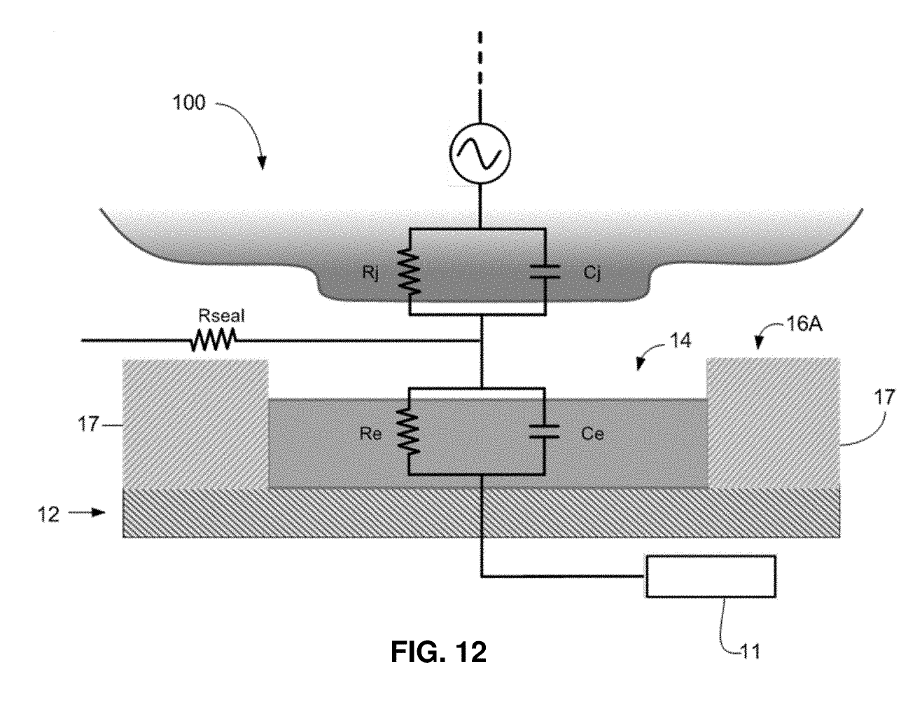

While FIG. 2 shows one embodiment of the microelectrode 10, other configurations of the substrate 12, the first electrode base 14 and the ridge 16 are also contemplated by the present disclosure. For example, FIG. 12 shows another embodiment of the present disclosure where a ridge 16A is formed by an adjacent material 17 that may be electrically insulating, or not. In some embodiments, the insulating material 17 is electrically non-conductive, which may also be referred to as dielectric. The adjacent insulating material 17 may at least partially surround the outer perimeter of the electrode base 14.

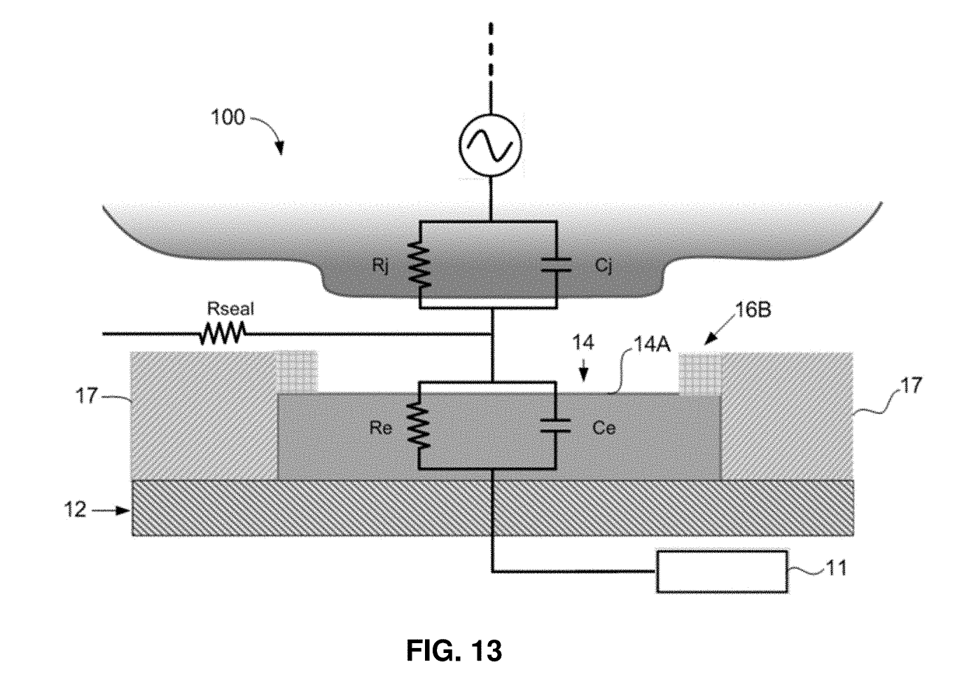

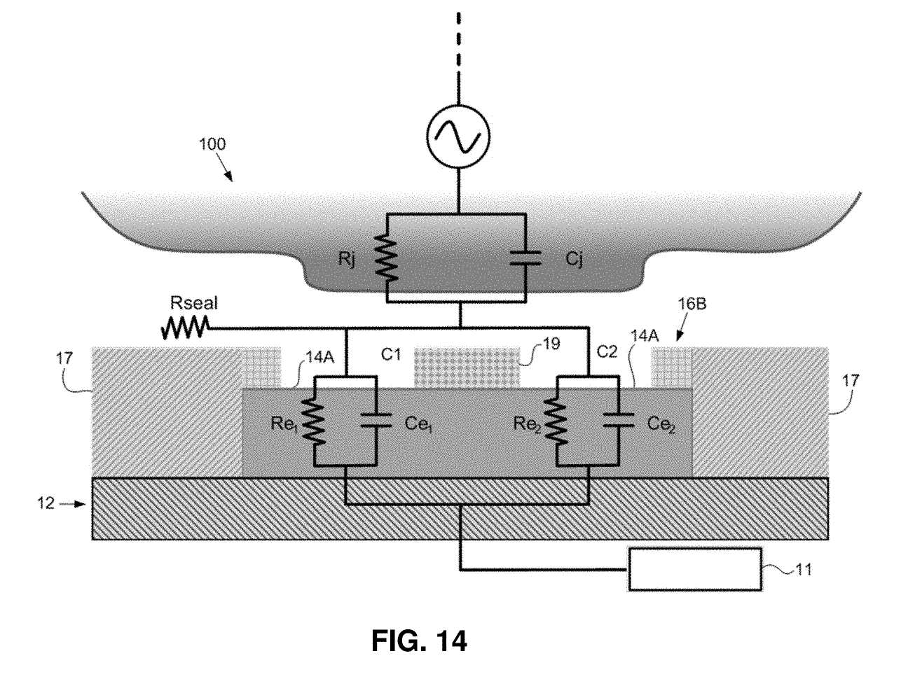

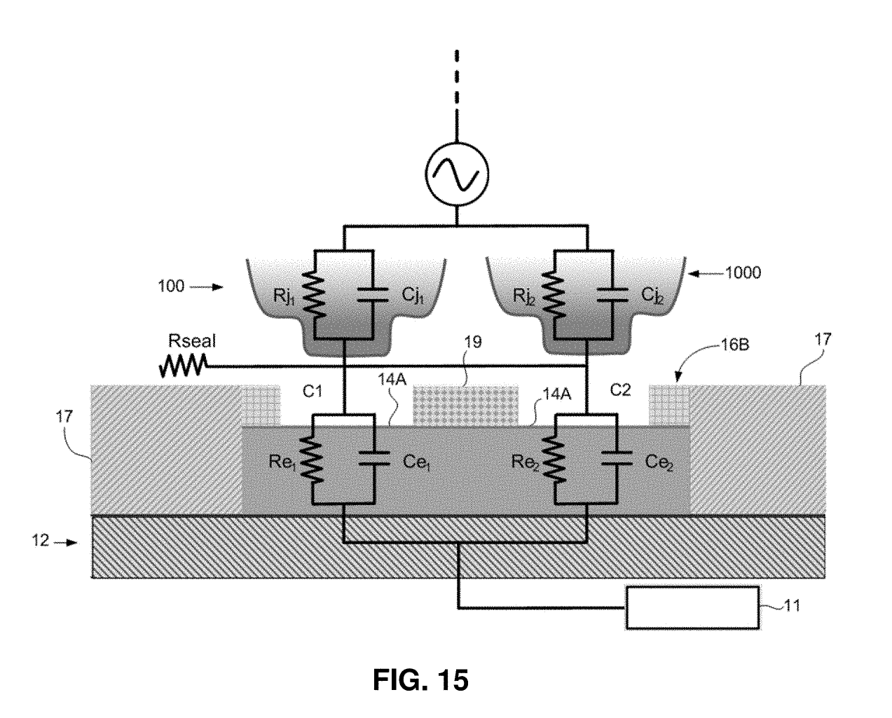

FIG. 13 shows another embodiment of the present disclosure where the insulating material 17 is positioned at least partially upon surface 14A to form a ridge 16B. FIG. 14 shows another embodiment of the present disclosure which is similar to the embodiment shown in FIG. 13 but with the addition of further insulating material 19 upon the surface 14A. The further insulating material 19 may then define a first channel C1 and a second channel C2 of the electrode base 14. Each channel defines a separate circuit in electrical communication with the electrode wire 14D and the adjacent electrically excitable cell. FIG. 15 shows the embodiment of FIG. 14 but there is one electrically excitable cell 100 that forms part of a circuit with the first channel C1 of the electrode base 14 and a second electrically excitable cell 1000 forms a second and separate electric circuit with the second channel. The phrase “ridge 16” may be used herein to generally refer to each of the ridges 16, 16A and 16B, unless specified otherwise.

Regardless of the specific embodiment, the microelectrode 10 is positionable at an interface with an electrically excitable cell. By this positioning, the ridge 16 is positionable opposite to a portion of the electrically excitable cell so that the ridge 16 engulfs at least a portion of the electrically excitable cell's outer membrane. The engulfing of at least one portion of the cell membrane by the ridge 16 may mimic the configuration of two neurons at some forms of naturally occurring synapses. In these naturally occurring synapses a post-synaptic terminal engulfs a portion if not all of the pre-synaptic terminal. Without being bound by any particular theory, the engulfing configuration that mimics some natural synapses may strengthen the signal at the post-synaptic terminal by generating one or more, larger amplitude post-synaptic potentials, which is an important process for trans-synaptic communication between two neural cells that are separated by a synaptic cleft.

Current can be propagated along neurons by a phenomenon that is referred to as an action potential. Typically, action potentials are propagated by a flow of ions through ion channels located in the neural cell membrane, this being due to an electro-chemical gradient variation between the inside and outside of the neural cell membrane. When the action potential propagates along the neural cell and arrives at the synaptic cleft, the action potential induces a release of chemical neurotransmitters from the neural cell 100A into the synaptic cleft. The neurotransmitters diffuse across the synaptic cleft and bind to receptors on the post-synaptic neuron 100B, causing localized changes in the levels of ions.

Recording of electrical activity using extracellular recording techniques results from electron movement that causes a potential difference in local trans-membrane charges at the first base surface 14A of the electrode base 14. In this fashion, the electrode base 14 is configured to detect electrical signals from one or more adjacent electrically excitable cells. In some embodiments, these electrical signals may be in the form of ionic fluxes across the adjacent electrically excitable cell's membrane.

Embodiments of present disclosure provide the microelectrode 10 that detects changes in the levels of various ions within the local environment that may be caused by action potentials or ionic fluxes that occur within the interface with an adjacent electrically excitable cell. The changes in ion levels affect the electric charges present at the surface of an individual electrode base 14 within the microelectrode 10. Electric charges present on the electrode base 14 then generate an electrical signal in the form of either a current or voltage that is transmitted via the electrode wire 14D. Without being bound by any particular theory, the engulfing by the ridge 16 of at least one portion of an adjacent electrically excitable cell may permit the microelectrode 10 to achieve a desired single-to-noise ratio because the changes in ions levels will be localized at least partially by the ridge 16 and the effect of those changes may be concentrated onto the first base surface 14A of the electrode base 14. Said another way, the ridge 16 may increase the sealing resistance (Rseal) at the interface between the microelectrode 10 and the adjacent electrically excitable cell or cells.

In another embodiment of the present disclosure, the microelectrode 10 is electronically connected to an electronic system 11. The electronic system 11 may comprise a pre-amplifier and an amplifier for amplifying and transmitting the electric signal that is detected at the interface to an analysis and recording system. In some embodiments of the present disclosure, the analysis and recording system of the electronic system 11 may be a computer with associated analysis and recording software. In one embodiment of the present disclosure the electronic system 11 may comprise an MEA1060; Multichannel System (Reutlingen, Germany) or a comparable electronic recording system.

Embodiments of the present disclosure provide the microelectrode 10 that may be fabricated using a bottom-up fabrication method, a top-down fabrication method or any combination of both. A prevailing factor in selecting a suitable fabrication method may be the ability to make the ridge 16 in a controlled and repeatable manner.

One example of a top-down fabrication process is the photolithography process during which layers of a thin material are deposited onto the substrate 12, for example upon the first surface 12A. Then a portion of the deposited materials are selectively removed to form the desired features of the microelectrode 10, such as the electrode base 14 and the ridge 16. There are different types of photolithography methods that may be suitable to make the microelectrode 10, including but not limited to: standard optical lithography, nano-lithography, lift off, etch back and combinations thereof.

There are also different bottom-up fabrication methods that may be suitable to make the microelectrode 10 and the ridge 16. In general, a bottom-up fabrication method places atoms or molecules one at a time to build the desired nanostructure. A typical bottom-up fabrication method is to deposit or grow the material that defines the electrode base 14 and the ridge 16 thereon.

Several methods are suitable to accomplish deposit materials, such as but not limited to the following:

Physical vapor deposition (PVD) uses physical process to produce a vapor of the material, which is then deposited on the target object. Examples include but are not limited to: cathodic arc deposition, electron-beam physical vapor deposition, evaporative deposition, pulsed laser deposition or sputter deposition.

Chemical vapor deposition (CVD) is a chemical process in which the substrate is exposed to one or more volatile precursors, which react and/or decompose the substrate surface to produce the desired material. Examples include but are not limited to: atmospheric pressure CVD (APCVD), low-pressure CVD (LPCVD), ultrahigh vacuum CVD (UHVCVD), aerosol assisted CVD (AACVD), direct liquid injection CVD (DLICVD), microwave plasma-assisted CVD, Plasma-Enhanced CVD (PECVD), remote plasma-enhanced CVD (RPECVD), atomic-layer CVD (ALCVD), combustion chemical vapor deposition (CCVD), hot filament CVD (HFCVD), hybrid physical-chemical vapor deposition (HPCVD), metal organic chemical vapor deposition (MOCVD), rapid thermal CVD (RTCVD), vapor-phase epitaxy (VPE) or photo-initiated CVD (PICVD).

Epitaxy, casting, oxidation, electro-chemical deposition, chemical or physical self-assembly, Sol-gel technology, and 3-D printers all may also be suitable fabrication methods.

Direct patterning of the deposited or grown material can be accomplished by chemical etching, laser processing, ultraviolet (UV) light, electron-beams, x-rays or other means. Patterning can also be accomplished by direct manipulation of the material at the nano-scale, for example via atomic force microscopy (AFM) or scanning tunneling microscopy (STM).

Because electrically excitable cells can be of different sizes, as within a species and between species, one of skill in the art will appreciate that the substrate 12, the electrode base 14 and the ridge 16 may be fabricated to a variety of specific dimensions, using different materials, coatings and surface features to suit the desired application. For example, the substrate 12 may be made from ceramic, silicon, glass, plastic, as a printed circuit board or combinations thereof. The electrode body 14 may be made from gold, titanium, iridium, other suitable metals or combinations thereof. The electrode body may be coated in chemical coatings such as platinum black prior to forming the ridge 16, which may also be referred to as extending the ridge 16. The chemical coatings may increase the signal-to-noise ratio of signal generated by the microelectrode 10. The ridge 16 itself may be made of conductive or dielectric materials (insulating material). Furthermore, the surface 14A may be textured or patterned, which results in a roughened surface with an increased surface area. The textured surface 14A may potentially enhance adhesion of the adjacent electrically excitable cell to the microelectrode 10, which also may enhance the signal-to-noise ratio of signal generated by the microelectrode 10.

### EXAMPLES

### Example 1

### Fabricating and Characterizing an Example Electrode

One example of the microelectrode 10 includes multiple electrodes 14 that are gold planar-electrodes 14 that were fabricated using a standard photolithography technique at the Advanced Micro/Nanosystems Integration Facility (AMIF) of the University of Calgary (Calgary, Alberta, Canada). Briefly, the example electrodes 14 were fabricated using a two-mask photolithography process on a substrate 12 of 49 mm×49 mm glass plate that was 1 mm thick. The example electrodes 14 were gold sputter deposited to a height of about 200 nm onto a 10 nm chromium adhesion layer. Using a standard photolithography process the sputtered gold and chromium formed an electrode base 14. Once the example electrode bases 14 were formed, the ridge 16 was added using an additional photolithography step. An epoxy-based photoresistive layer (SU8) with a thickness of about 5 μm was then deposited by spin-coating and patterned (or roughened) by photolithography to provide an insulation layer over the example electrode traces. Openings in the SU8 layer left the main microelectrode 10 bare for stimulation and or recording. In forming the microelectrode 10, the sizes and intervals between the example electrodes 14 can be adjusted by modifying the photomask designs. By using this fabrication technique the inventors demonstrated that they could maintain a relatively simple process that is not only cost sensitive but that is also adaptable.

Following fabrication of the example electrodes 14, the inventors characterized and validated the structure of the electrodes with atomic force microscopy (AFM). FIG. 3A and FIG. 3B provide the qualitative results from this AFM analysis.

FIG. 3A shows a three-dimensional representation of an example electrode base 14 at about a 40° tilt. The electrode base 14 has a diameter of about 30 μm. The ridge 16 is seen extending upwardly from the upper surface of the electrode along the perimeter of the electrode body 14C. The ridge 16 can also be seen continuing along the electrode wire 14D (bottom right of FIG. 3A). Having the ridge 16 along the wire and not limited to the circular area may increase the sealing resistance in a scenario where the electrically excitable cell 100 is not centered exactly opposite the electrode base 14. This arrangement also permits the manufacture of an increased area of the electrode base 14 that is in communication with the junctional membrane 102 of the neural cell 100.

FIG. 3B shows a cross-section of the example electrode base 14 that demonstrates the height and shape of the electrode, including the ridge. The example electrode base 14 was 30±1 μm in diameter, 200±15 nm in height, and the ridge varied between about 5 nm and about 15 nm in height and between about 2 to about 3 μm in width. The number 3 and 3′ correspond with the ends of the dotted line shown in FIG. 3A.

### Example 2

### A Custom MEA Layout Pattern and Neuron Cell Culture

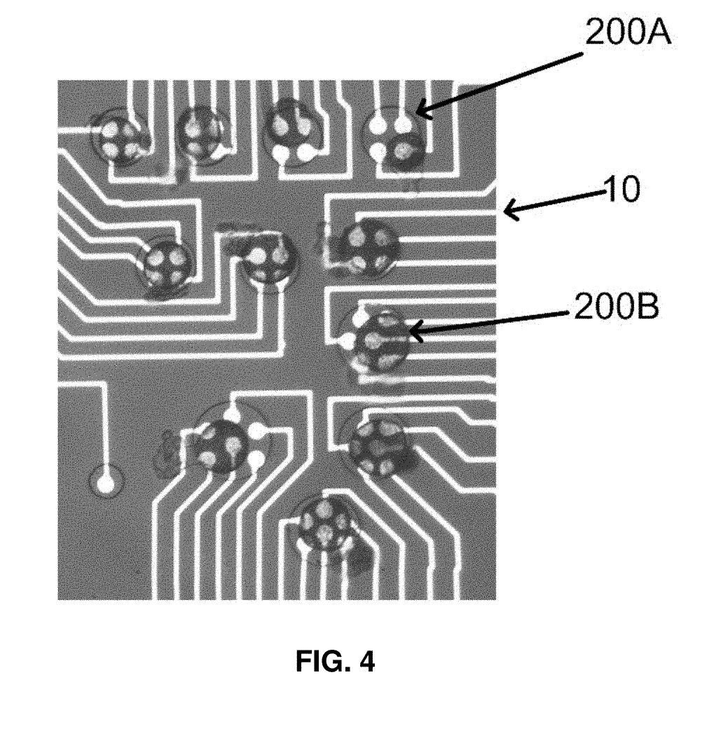

The electrode bases 14 produced in Example 1 were used in one embodiment of a layout pattern of an array of the microelectrode 10 (see FIG. 4). Neural cells were cultured on the microelectrode 10 and communication was established to interrogate the electrical activity of individual neural cells.

In order to overcome the challenges posed by the complexity of the mammalian neural network, the freshwater snail, Lymnaea stagnalis was used as a model system for the study of fundamental neuronal properties, synaptogenesis and network formation. This snail model provides larger neural cells with diameters about 30 μm to about 100 μm depending on the age of the animals, as compared to about 4 μm to about 10 μm for typical mammalian neurons. The structure and function of the cultured snail neural cells are also well characterized. Using this model, individual neural cells can be manipulated on the example array of microelectrode 10 with relative ease.

The neural cells were cultured according to the protocol described by Syed et al. (1990) In vitro reconstruction of the respiratory central pattern generator of the mollusk Lymnaea, Science 250:282-285, the entire disclosure of which is incorporated herein by reference. Briefly, the central ring ganglia was removed from 1 to 2 month old Lymnaea stagnalis snails and treated with trypsin (2 mg/mL; T-4665; Sigma-Aldrich, St Louis, Mo., USA). After about 20 minutes, a trypsin inhibitor (2 mg/mL; T-9003; Sigma-Aldrich) was applied for 15 minutes to stop the enzymatic reaction. For the purpose of these example experiments, the inventors isolated the specifically identified pre- and post-synaptic neurons, VD4, RPeD1 and LPeD1, by gentle suction applied through a fire-polished, SIGMACOTE®-treated glass pipette (SIGMACOTE is a registered trademark of Sigma-Aldrich Biotechnology L.P.). The neurons were then plated onto an array of microelectrodes 10 which were coated with poly-L-lysine in conditioned media (CM) that contained trophic factors that are necessary for growth and synapse formation. Trophic factors present in the conditioned media were obtained by incubating isolated Lymnaea stagnalis central ring ganglia from 2 to 6 month old animals for 3 to 7 days in a trophic-factor free, defined media that is commercially available as DM; L-15 Special Order (Life Technologies, Gaithersburg, Md., USA). The neurons were allowed to settle overnight and used for experiments the next day at about 12 to about 18 hours post culture.

The cultured neurons were individually placed on a set of multiple microelectrodes 10 (4 or 6 microelectrode 10 per set). An example of four individual microelectrodes 10 with one or more neurons cultured thereon is indicated in FIG. 4 as 200A. An example of six individual microelectrodes 10 with one or more neurons cultured thereon is indicated in FIG. 4 as 200B. Each individual microelectrode 10 in FIG. 4 has an electrode body 14C with a substantially circular geometry and an individual electron wire 14D that extends away from the electrode body 14C. This configuration permitted the inventors to record neuron activity continuously even if a given neuron had moved away from an initial culture site as described in Wij denes, P. et al., (2016) A novel bio-mimicking, planar nano-edge microelectrode enables enhanced long-term neural recording. Scientific Reports 6, Article number: 34553, the entire disclosure of which is incorporated herein by reference.

The cultured neurons grew on the microelectrodes 10 at a rate of up to 1 mm per 24 hours. This growth may indicate a degree of biocompatibility with the substrate materials used in the fabrication process. When pre- and post-synaptic neurons (VD4 and LPeD1, respectively) were cultured together in a paired, soma-soma configuration, action potentials could be triggered in the pre-synaptic neuron using intracellular sharp electrodes which elicited 1:1 excitatory post-synaptic potentials (EPSPs) of a constant amplitude and latency. Approximately 95% of the paired neurons formed a synapse within 24 hours on the array of microelectrodes 10.

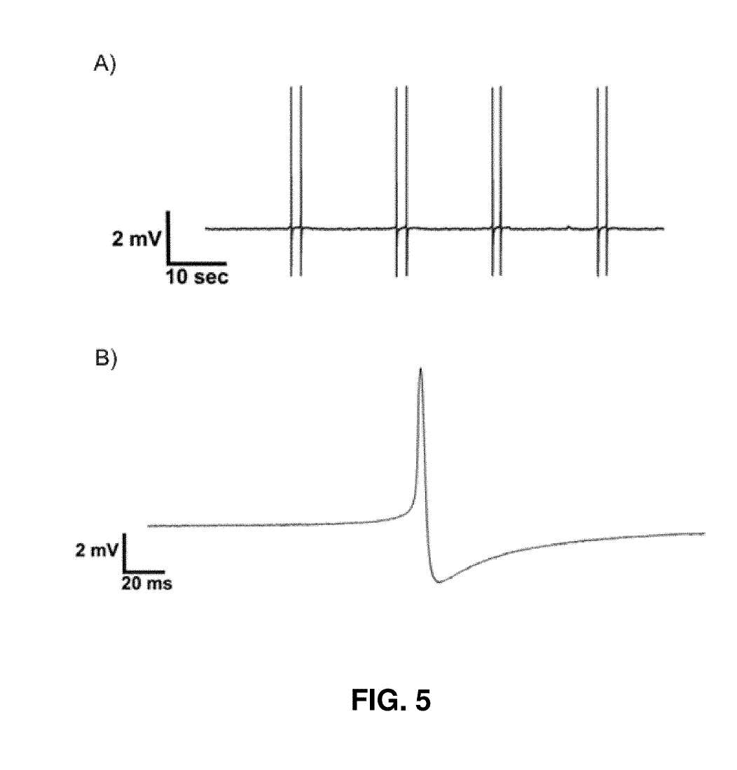

Using the example array of microelectrode 10, action potentials were recorded with up to 10.6 mV amplitude, peak-to-peak (FIG. 5A and FIG. 5B). FIG. 5A shows an example action-potential recording from a single neuron that showed a characteristic pattern of neuronal electrical activity. FIG. 5B shows an example of a single action potential that was recorded with a clearly defined depolarization phase that is followed by a rebound, hyperpolarization phase.

The neurons' coupling coefficient was calculated to be 0.15, which is 15 times higher than what has been reported with traditional planar and resistor electrodes by Spira, M. E. and A. Hai, (2013) Multi-electrode array technologies for neuroscience and cardiology. Nat Nanotechnol, 8(2): p. 83-94, the entire disclosure of which is incorporated herein by reference. This data may support the use of the array of microelectrodes 10 to study patterned activities of single cells, which may be a key step in studying neural network connectivity over long time periods.

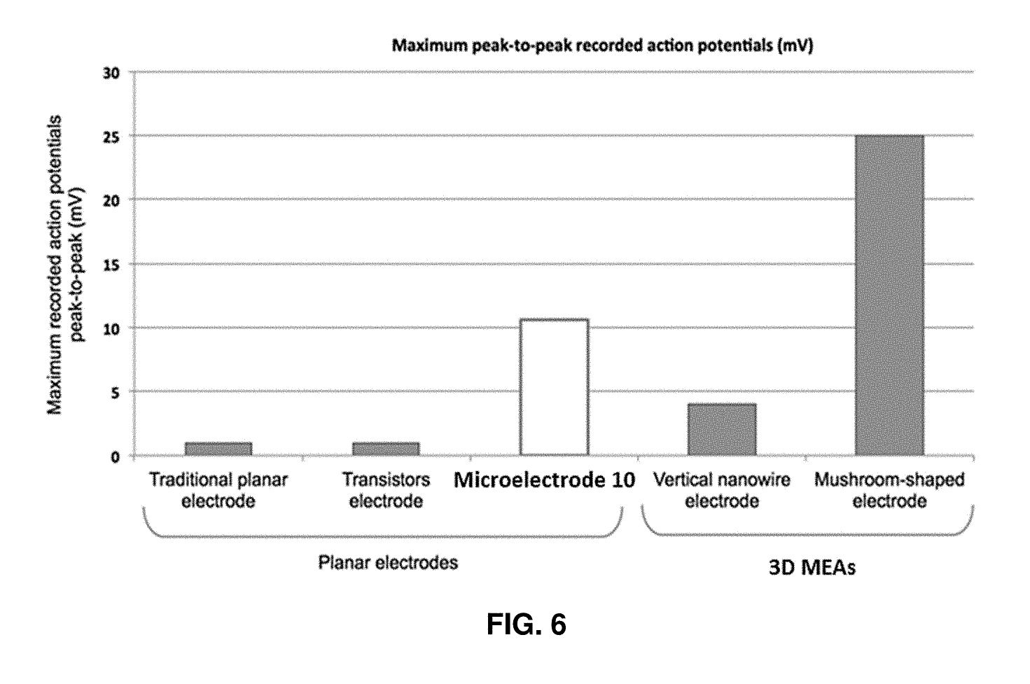

FIG. 6 is a bar graph that shows a comparison of the maximum peak-to-peak amplitude of action potentials that were recorded with some example planar electrodes, the array of microelectrodes 10 with the ridge 16 and example 3D-MEAs.

In comparison with planar electrodes that can record neural activity for several months, 3D-MEAs can only monitor activity for a limited period of time with a maximum of 2 days currently reported in the literature (for example see Spira, M. E. and A. Hai, (2013) Multi-electrode array technologies for neuroscience and cardiology. Nat Nanotechnol, 8(2): p. 83-94, the entire disclosure of which is incorporated herein by reference. Without being bound by any particular theory, this limitation is not likely due to biocompatibility issues as most known devices use materials that are conducive to neural growth and network formation. A possible reason for this limitation may lie in a disrupting of the cell's three-dimensional environment. Cultured neural cells are not static, they tend to move on top and around the electrodes, pulled by growth cones and newly formed neurites. When three-dimensional components restrain this natural movement (e.g. 3D-MEAs with spike or mushroom-shaped electrodes), neural cells may experience membrane rupture, cytosol leakage and death. The embodiments of the present disclosure that comprise one or more microelectrodes 10 with the ridge 16 may restrict neural cell movement less than 3D-MEAs with spikes and mushrooms. The limited neural cell restriction may have contributed to the ability to record neuron activity for more than a month.

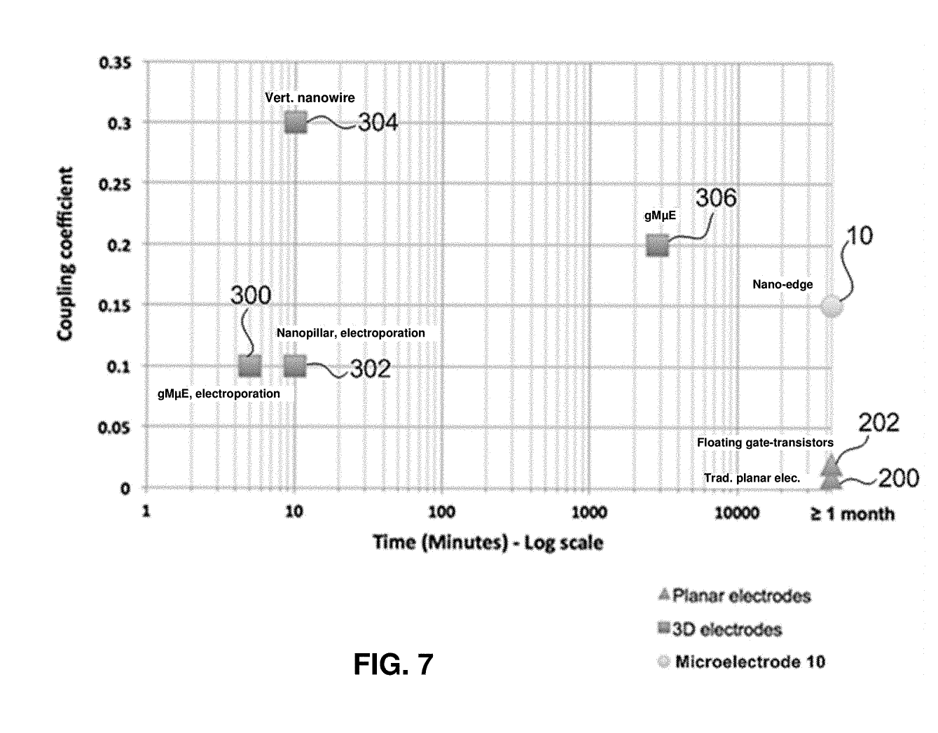

FIG. 7 depicts the coupling co-efficient versus the time period for capturing viable neural cell activity recordings for some example planar electrodes, 3D MEAs and an embodiment of the microelectrode 10 with the electrode base 14 with the ridge 16 of this disclosure. The most commonly reported micro-/nano-electrodes were compared to record neural activity in vitro and used the maximum coupling coefficient and the longest reported recording time to evaluate electrode capabilities. The comparison included: planar microelectrodes (shown as triangles in FIG. 7) such as traditional planar electrodes 200 and floating gate-transistors 202; 3D microelectrodes (shown as squares in FIG. 7) such as gMμE electrophoresis electrodes 300, nano-pillar electrophoresis electrodes 302, vertical nanowires 304 and gMμE electrodes 306. The microelectrode 10 (shown as a circle in FIG. 7) can record action potentials with a coupling coefficient comparable to 3D-MEA electrodes for a period of time equivalent to traditional planar electrodes (shown as triangles in FIG. 7).

### Example 3

### Computer Simulation

To determine any effects of the ridge 16 on the physical properties of the example electrode base 14, a computer model simulation was conducted to confirm recording efficacy observed. The observed efficacy can be attributed to two main factors: (i) a decrease in the electrode impedance, or (ii) an increase of the sealing resistance.

A neuron-electrode interface was modeled using the Electric Currents software module in COMSOL Multiphysics (COMSOL Inc., Burlington Mass.). The goal of the simulation was to determine the effect of the ridge 16 on the sealing resistance. The sealing resistance is defined as the resistance that restricts leakage of current through a gap at the interface between a neural cell and the substrate. The computer model simulation was adapted from previous models of neuron simulation described in Ghazavi, A. et al. (2015) Effect of planar microelectrode geometry on neuron stimulation: finite element modeling and experimental validation of the efficient electrode shape, J Neurosci Methods, 248: p. 51-8 and Buitenweg, J. R., W. L. Rutten, and E. Marani, (2000) Finite element modeling of the neuron-electrode interface, IEEE Eng Med Biol Mag, 19(6): p. 46-52, the entire disclosures of which are incorporated herein by reference.

Briefly, a glass substrate and extracellular fluid were modeled as infinite boundaries. The electrode height was set at 200 nm and its width was set to 30 μm. The neural cell was positioned 50 nm above the electrodes to echo the gap found in neural cell-electrode interfaces and was modeled with diameters from 5 μm to 80 μm which is representative of most vertebrate and invertebrate neuron diameters. Finally, the ridge 16 was modeled at various heights from 0 nm, which is similar to traditional planar electrodes without a ridge, to 50 nm which is about the same height as the gap between the electrode and the neural cell.

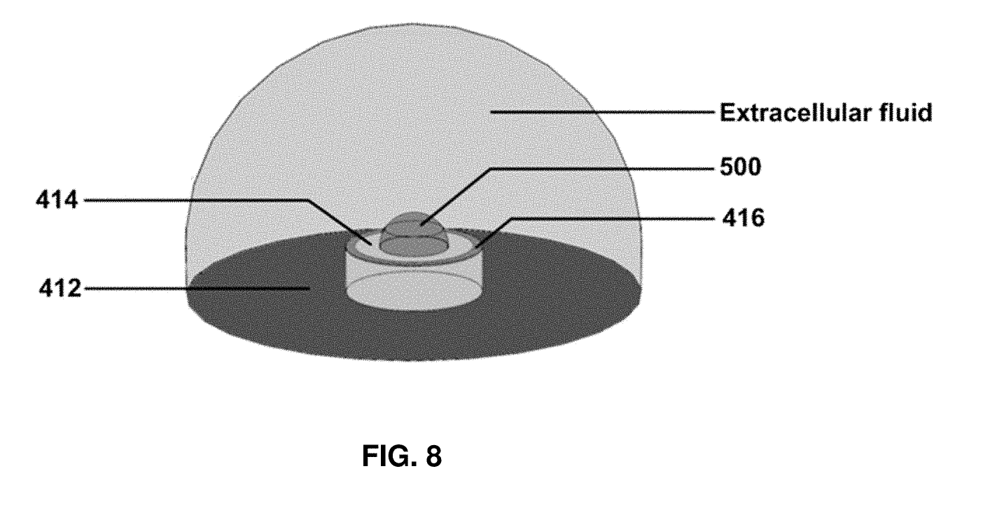

As shown in FIG. 8, the computer model simulation consists of several domains. Firstly, a glass substrate 412, which acts as an insulating layer, was modeled in the simulation and forms an electrode base 414 of a microelectrode 410 according to embodiments of the present disclosure. The glass substrate 412 was modeled with an infinite boundary for this simulation. The electrode base 414 was modeled above the substrate 412 using a gold cylinder with a diameter of 30 μm, which reflects the size of the electrode bases 414. To properly reflect the fabricated electrode bases 414, a thin layer of chromium was inserted in between the electrode base 414 and the substrate 412, which acts as an adhesive between the two layers. However, there were no differences observed in the results when the chromium layer was added or removed from the model. The boundaries and volume of an adjacent electrically excitable cell 500 were simulated using a semi-circle which acted as the membrane and intracellular fluid, respectively. A 2 μm wide ridge 416, which is referred to as a nano-edge in FIG. 8 (and Table 1 below) was added to the electrode via a ring of dielectric material around the upper edges of the electrode base 414. While the height of the fabricated ridge 416 ranged between 5 nm to 15 nm, a ridge 416 was simulated with a height that ranged from 0 nm to 50 nm to provide a better understanding of any ridge-related effect. The remaining external volume was filled with extracellular fluid. Similar to the substrate 412, this domain of extracellular fluid was modeled as an infinite region. Table 1 below shows the values of electrical conductivity and relative permittivity used for the various materials.

TABLE 1
Electrical Conductivity and Relative Permittivity Values
Used in the Computer model simulation.

| Materials | Electrical Conductivity | Relative Permittivity |
|---|---|---|
| Gold electrode | 45.6e6 | 6.9 |
| Cell Membrane | 7.93e−8 | 5.6470 |
| Extracellular Fluid | 0.84 | 80 |
| Intracellular Fluid | 0.68 | 80 |
| Dielectric nano-ridge | 3.5e−15 | 4.1 |

Two specific meshes were used in the computational simulation model to improve the result outcomes and analysis. A standard free tetrahedral mesh was used for the neural cell and the surrounding extracellular fluid. However, the free tetrahedral mesh was unable to mesh the smaller portions of the simulation due to computational limitations with regards to the smaller elements. Therefore, a free triangular swept mesh was implemented in those parts. This mesh was utilized for the substrate 412, electrode base 414, and the thin layers in between the electrode base 414 and the cell 500. A swept mesh may be better for modeling thin layers and non-proportioned domain sizes by avoiding redundant mesh elements, which also decreases the computation time. The mesh contained between 253,178 and 157,401 mesh elements. Increasing the mesh from 250,513 to 779,642 elements resulted in a very small change of 0.02 MΩ to the sealing resistance, this may mean that a larger mesh did not have an extensive impact on the results. Therefore, a smaller mesh was used to reduce computational time. The sealing resistances calculated using this model for the electrode base 14 with a 30 μm diameter ranged from 0.66 MΩ to 8.71 MΩ depending on the height of the ridge 16 and the size of the neural cell simulated. When analyzing the sealing resistance values of planar electrodes without any ridge 416, the results were in the same range as reported for transistor planar electrodes by Cohen et al. (2008), Reversible transition of extracellular field potential recordings to intracellular recordings of action potentials generated by neurons grown on transistors, Biosens Bioelectron, 23(6): p. 811-9.

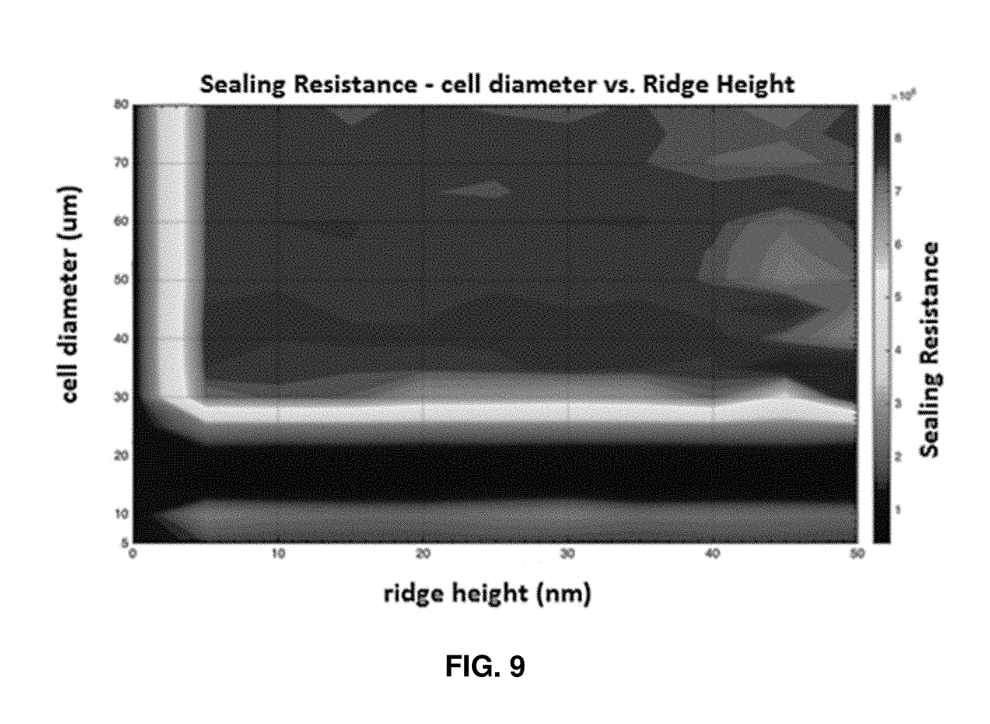

FIG. 9 shows an example of heat-map results from the computer model simulation for the sealing resistance as a function of the neural cell's 500 diameter and the ridge 416 height. A rapid increase in sealing resistance was noted when the ridge 416 is present and the neural cell diameter is equal or larger than the electrode base 414 (here 30 μm in diameter).

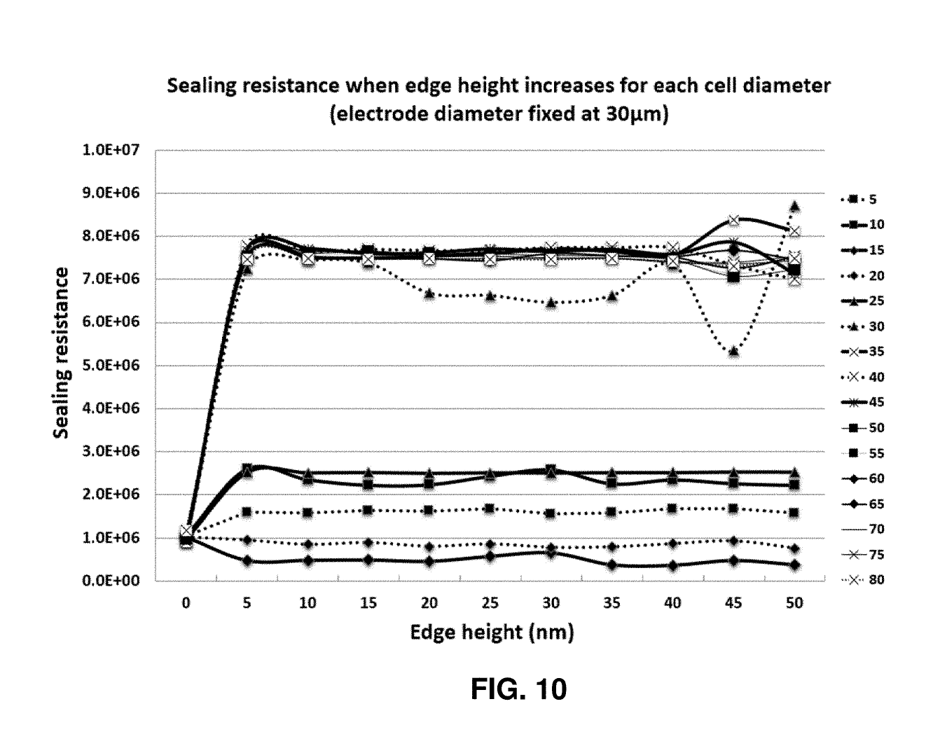

FIG. 10 shows example sealing-resistance results from the computer model simulation for each neural cell diameter when the ridge 416 increased in height. When the neural cell diameter reaches a diameter equal or larger than the electrode base 414 and when a ridge 416 is present, the sealing resistance reached a plateau of 7.49±0.34 MΩ for average.

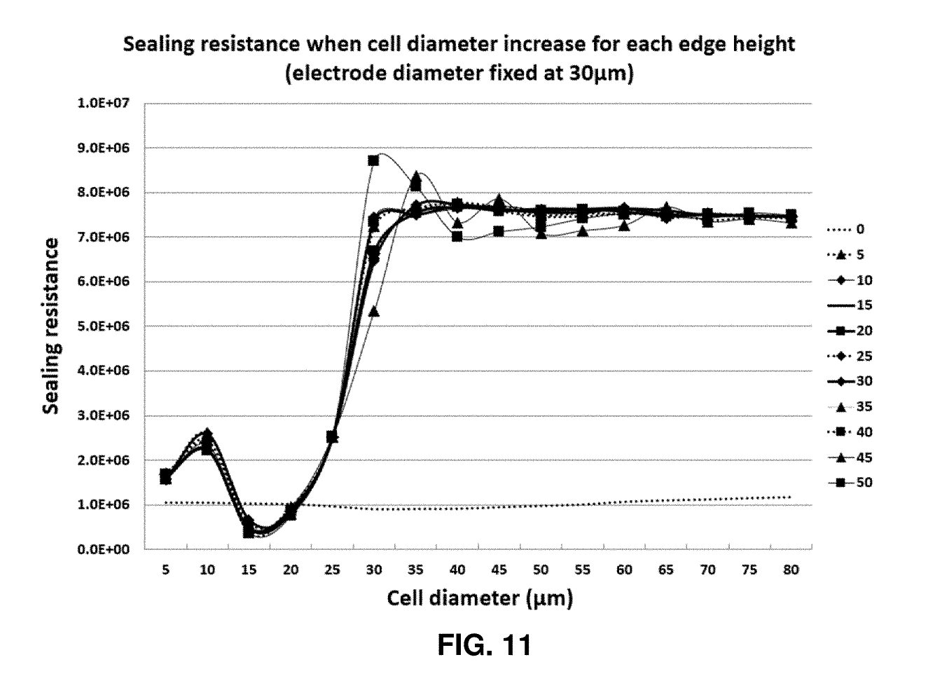

FIG. 11 shows example sealing-resistance results from the computer model simulation for each ridge 416 height as the neural cell diameter increases.

FIG. 16 shows a line graph of sealing resistance versus ridge width data that was obtained using the computer simulation model. In this analysis the diameter of the adjacent electrically excitable cell was 10 μm, the electrode base 414 diameter was 30 μm and the height of the ridge 414 was 5 nm. Without being bound any particular theory, the data in FIG. 16 may represent a model of a mammalian neuron.

Without being bound by any particular theory, the diameter of the adjacent electrically excitable cell may be a relevant factor for the observed sealing-resistance value, which significantly increases when the adjacent electrically excitable cell has a diameter equal to or larger than the electrode base 14 diameter or width. When the adjacent electrically excitable cell's diameter is smaller than the diameter or width of the electrode base 414, the sealing-resistance values tend to vary due to current leakage.

If a ridge 416 is present with a height that is greater or equal to 5 nm and the adjacent electrically excitable cell's diameter is equal to or larger than the width or diameter of the electrode base 414, the sealing resistance remains approximately the same with an average of 7.49±0.34 MΩ. This is independent of any increase of the height of the ridge 416 above 5 nm. Without the ridge 416, no significant difference in sealing-resistance vales were observed (average of 1.03±0.08 MΩ), which is independent of the adjacent electrically excitable cell's diameter. Therefore a ridge 416 with a height of about 5 nm may be better suited for biological recordings as it decreases the risk of physically damaging any adjacent electrically excitable cell.

I claim:

1. A microelectrode for communicating with an electrically excitable cell, the microelectrode comprising:
(a) a body with a perimeter;
(b) an electrode wire that is electronically connected to the body and that is electronically connectible to an electronic system; and
(c) a ridge that extends away from the perimeter of the body for increasing a sealing resistance value between the electrode and the electrically excitable cell.

2. The microelectrode of claim 1, wherein the ridge comprises an electrically conductive material or an electrically non-conductive material.

3. The microelectrode of claim 1, wherein the ridge is formed from an electrically non-conductive material that is adjacent to the body.

4. The microelectrode of claim 3, wherein the electrically non-conductive material is at least partially positioned upon the body.

5. The microelectrode of claim 1, wherein the perimeter is substantially circular from a top-plan perspective.

6. The microelectrode of claim 1, wherein the perimeter is substantially non-circular from a top-plan perspective.

7. The microelectrode of claim 1, wherein the body further comprises an interfacial surface and the ridge extends away from the interfacial surface.

8. The microelectrode of claim 1, wherein the body further comprises an interfacial surface that is textured for increasing a surface area of the body.

9. The microelectrode of claim 1, wherein the body further comprises a chemical coating.

10. The microelectrode of claim 1, wherein the body further comprises further insulating material for defining at least a first and a second channel of the microelectrode.

11. The microelectrode of claim 10, wherein the first channel and the second channel each define a separate electric circuit with the electrically excitable cell.

12. The microelectrode of claim 10, wherein the first channel defines an electric circuit with the electrically excitable cell and the second channel defines a second electric circuit with a second electrically excitable cell.

13. A microelectrode array for communicating with one or more electrically excitable cells, the microelectrode array comprising:
(a) two or more microelectrodes, each microelectrode comprising:
(i) a planar body with a perimeter;
(ii) an electrode wire that is electronically connected to the body and that is electronically connectible to an electronic system; and
(iii) a ridge that extends away from the perimeter of the planar body for increasing a sealing-resistance value between the electrode and the one or more electrically excitable cells.

14. A method for fabricating a microelectrode comprising steps of:
(a) providing a substrate with a first surface;
(b) positioning an electrode base upon the first surface, the electrode base comprising a first base surface that is opposite to the first surface; and
(c) forming a ridge that extends away from the first base surface, wherein the ridge is for increasing a sealing-resistance value between the electrode and the one or more electrically excitable cells.

15. The method of claim 14, wherein one or both of the positioning step and the forming step are performed by at least one of a top-down method, a bottom-up method and a combination thereof.

16. The method of claim 15, wherein the top-down method is selected from a group consisting of standard optical lithography, nano-lithography, lift off, etch back and combinations thereof.

17. The method of claim 15, wherein the bottom-up method is one or both of a physical vapor deposition method and a chemical vapor deposition method.

18. The method of claim 14, wherein one or both of the positioning step and the forming step are performed by at least one of epitaxy, casting, oxidation, electro-chemical deposition, chemical self-assembly, physical self-assembly, sol-gel technology and 3-D printing.

19. The method of claim 14, further comprising a step of patterning one or both of the electrode base and the ridge, wherein the step of patterning is accomplished by at least one of chemical etching, laser processing, ultraviolet light, electron beams, x-rays, atomic force microscopy manipulation and scanning tunneling microscopy manipulation.

20. The method of claim 14, further comprising a step of coating the electrode base with a chemical coating, wherein the coating step occurs prior to the forming step.
